(19) United States
(12) Patent Application Publication
(10) Pub. No.: US 2017/0258621 A1
(43) Pub. Date: Sep. 14, 2017

(54) APPARATUS AND METHODS FOR CERVICAL TRACTION

(71) Applicant: Core Products International, Inc., Osceola, WI (US)

(72) Inventor: Paul Norstrem, Osceola, WI (US)

(21) Appl. No.: 15/456,141

(22) Filed: Mar. 10, 2017

Related U.S. Application Data
(60) Provisional application No. 62/307,070, filed on Mar. 11, 2016.

(51) Int. Cl. A61F 5/048

(52) U.S. Cl. CPC A61F 5/048

(57) ABSTRACT

Apparatus and methods are provided for cervical traction of a subject. In an example, a cervical traction device can include a traction base, an anterior strap configured to apply pressure to a frontal bone of a subject's head and to couple to the traction base, and a posterior strap configured to engage an occipital bone of the subject and to couple to the traction base, wherein the posterior strap includes a tubular portion configured to engage the occipital bone.

### CLAIM OF PRIORITY AND RELATED APPLICATIONS

This application claims the benefit of priority under 35 U.S.C. 119(e) to Norstrem, U.S. Provisional Patent Application No. 62/307,070, filed on Mar. 11, 2016, and titled, “APPARATUS AND METHODS FOR CERVICAL TRACTION,” which is hereby incorporated by reference herein in its entirety.

### TECHNICAL FIELD

The disclosure herein relates generally to traction devices and more particularly to an improved straps and anchor locations for cervical traction devices.

### BACKGROUND

Head harnesses are typically used in conjunction with a variety of devices that attempt to generate a traction force so as to apply cervical traction therapy to a subject. In certain situations, cervical traction can successfully treat many disorders of the neck and spine. Current designs attempt to focus the traction force on the occipital bone by applying a counter force to the frontal bone or to the mandible of a subject. The accepted method of applying these forces is the use of flexible straps designed to fit over the described area of the subject. The former technique commonly causes the head to move forward into a state of flexion, as the frontal bone strap is anchored at the same point as the occipital bone strap, thus allowing rotational force to occur rather than the desired linear traction force. Placing the head in a state of flexion causes the typical occipital lobe strap to fail engagement with the occipital bone. The latter method usually focuses more force on the mandible than the occipital bone. Both of these techniques cause undesirable and often unacceptable forces on the subject contrary to the intention of the traction therapy.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 illustrates general the use of a cervical traction device according to an example of the present subject matter on a subject in a supine position.

FIG. 2 illustrates generally an anterior strap for a cervical traction device according to an examples of the present subject matter.

FIGS. 3A and 3B illustrate generally a posterior strap for a cervical traction device according to an example of the present subject matter.

FIG. 4 illustrates generally strap hardware for securing and adjusting a strap of a cervical traction device.

FIG. 5 illustrates generally use of a cervical traction device according to an example of the present subject matter on a subject in an upright position.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

The present inventors have recognized improved apparatus and methods, including improved straps, for cervical traction devices. In certain examples, an improved posterior cervical device strap can include structural features that allow the strap to better engage the occipital bone of a subject than the relatively flat straps of current cervical traction devices. In some examples, a separate anchor for a frontal lobe strap, or anterior strap, is also provided. The improved anchor location for the anterior strap can eliminate undesirable rotational force during cervical traction. In certain examples, the combination of the improved straps and the improved location of the anchor points for the anterior strap can allow the head of a subject to remain in a position considered neutral to mild extension during traction, which is considered ideal for cervical traction therapy. In certain examples, techniques according to the present subject matter can reduce or eliminate forward or rotational motion of the head into a state of flexion and can promote a. linear traction force along a line generally parallel with the subject's spine. Thus, such techniques use a linear traction force to provide spinal decompression as opposed to inducing an undesirable rotational force on the spine.

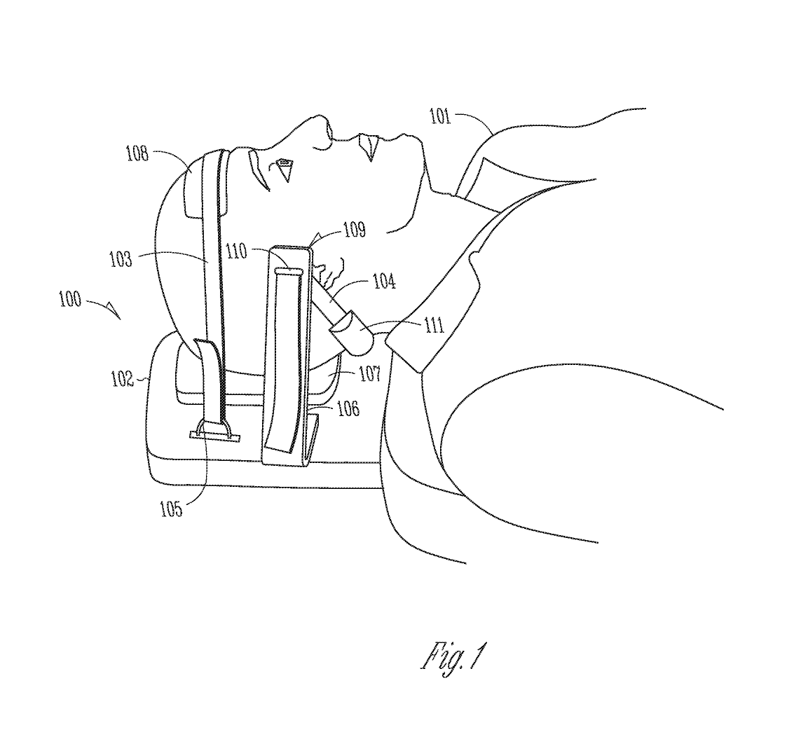

FIG. 1 illustrates general the use of a cervical traction device 100 according to an example of the present subject matter on a subject 101 in a supine position. In certain examples, the cervical traction device 100 can include a traction base 102, an anterior strap 103 and a posterior strap 104. The traction base 102 can provide a rigid structure to anchor forces applied to the subject 101 via the straps 103, 104. In certain examples, the traction base 102 can include left and right 105 anterior strap anchors, left and right 106 posterior anchor structures, and one or more optional cushions 107 to support the back of the head of a subject 101.

The left anterior strap anchor (not shown) and the right anterior strap anchor 105 can allow an anterior strap 103 to be laid across the frontal bone of the subject 101 and secured to the traction base 102. In certain examples, the anterior strap 103 can include hook and loop material (not shown) on a major surface of the strap 103 near each end of the anterior strap 103. Corresponding hook and loop material can be located on the same major surface closer to the center of the anterior strap 103, Each end of the anterior strap 103 can be secured to the corresponding left or right anterior strap anchor 105 by passing the end of the anterior strap 103 through the loop of the anchor 105 and folding the strap 103 back on itself and coupling together the hook and loop material at the end of the strap 103 to the corresponding hook and loop material located closer to the center of the strap 103. In certain examples, the anterior strap 103 can include a comfort pad 108 that, in certain examples, can be positioned between the strap 103 and the subject's skin. In certain examples, the traction base 102 can include multiple pairs of anterior strap anchors 105 to accommodate subjects with different sized heads. In some examples, a single pair of anterior strap anchors 105 can provided and can be adjustable to accommodate the size and shape of the head of each individual subject.

The left posterior anchor structure (not shown) and the right posterior anchor structure 106 can allow the posterior strap 104 to be positioned and secured such that the posterior strap 104 provides an appropriate cervical traction force. The posterior anchor structures 106 can he mechanically coupled to the traction base 102 and can provide an anchor position 109 near the subject's temples such that the posterior strap 104 can exert a traction force that can stretch the neck muscles, provide a separation force to the vertebra at the top of the spine, open the nerve channels of the spine near the neck or combinations thereof. In some examples, the posterior anchor structures 106 can be adjustable. In some examples, the posterior anchor structures 106 can be adjustable along an axis parallel to the subject's spine. In some examples, the posterior anchor structures 106 can be adjustable along an axis perpendicular to the subject's spine. In some examples, the posterior anchor structures 106 can be adjustable along an axis perpendicular to the subject's spine and along an axis perpendicular to the subject's spine to accommodate subjects of different size and shape.

In certain examples, each posterior anchor structure 106 can include an anchor apparatus for interfacing the posterior strap 104 with the posterior anchor structure 106. In some examples, the anchor apparatus can he a loop. In the illustrated example of FIG. 1, the anchor apparatus includes a slot 110 through which a corresponding end of the posterior strap 104 can be routed, Thus, the center portion 111 of the posterior strap 104 can be placed at the base of the occipital bone of the subject and the ends of the posterior strap 104 can be routed through the slots 110 of each posterior anchor structure 106. In certain examples, each end of the posterior strap 104 can include a length of hook and loop material. In some examples, a corresponding length of hook and loop material can be attached to an outer surface of each posterior anchor structure 106 and the posterior strap can be secured in place by mating the hook and loop material at the each end of the strap 104 with the hook and loop material attached to the corresponding posterior anchor structure 106. It is understood that strap hardware other than hook and loop material, loop anchors, and slots can be used to provide adjustable anchoring for the anterior strap 103 and the posterior strap 104 without departing from the subject matter of the present subject matter. It is also understood that the shape and size of the traction base 102 and the right and left posterior anchor structures 106 can differ from that shown in FIG. 1 without departing from the scope of the present subject matter.

In certain examples, the cervical traction device is positioned under the subject's head to position and secure the anterior strap 103 and the posterior strap 104 about the subject's frontal bone/forehead and occipital bone respectively. A force can then be applied to the traction base 102 in a direction away from the subject's torso such that a traction force is exerted on the subject's head via the traction base 102, anterior strap 103, and posterior strap 104.

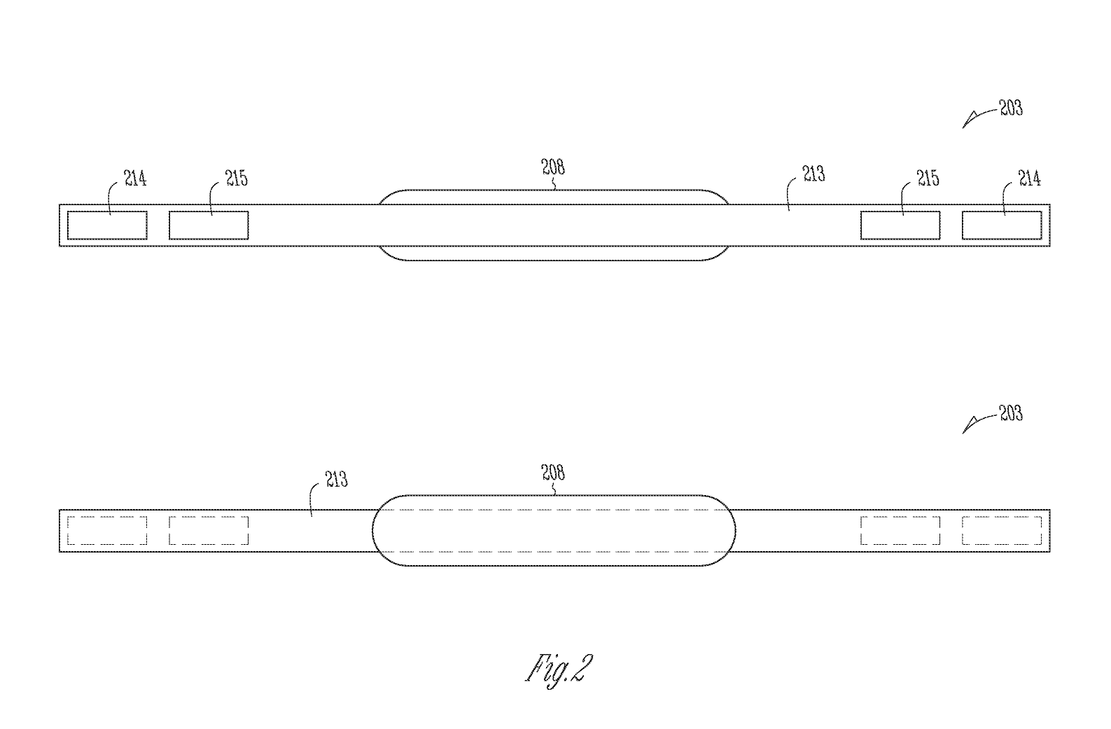

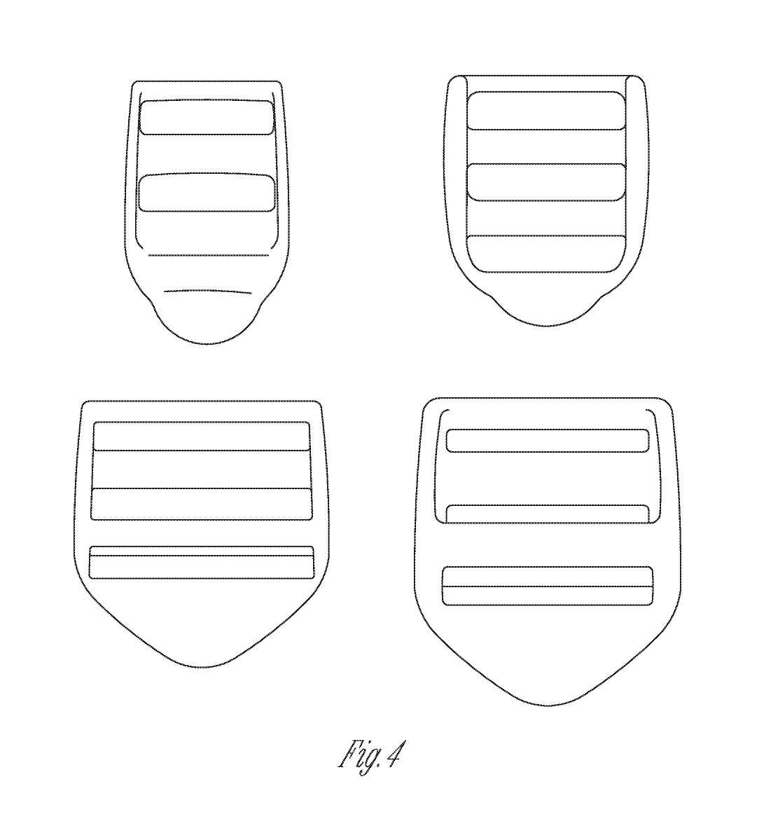

FIG. 2 illustrates generally an anterior strap 203 for a cervical traction device according to an examples of the present subject matter. The anterior strap 203 includes a length of web material 213 that can define the length of the anterior strap 203 and an optional cushion 208. The illustrated anterior strap 203 includes two areas 214, 215 of corresponding hook and loop material at each end. Such an arrangement of hook and loop material can allow an end of the strap with a first area of hook and loop material 214 to pass through a loop anchor and then fold back on the corresponding second area of hook and loop material 215 to secure the anterior strap 203 to the anchor. The lengths of the areas of hook and loop material 214, 215 can define the limits of adjustment of the anterior strap 203. In some examples, each end of the anterior strap can include only one area of hook and loop material and the anterior strap 203 can be secured using a second corresponding area of hook and loop material attached to the cervical traction device. In some examples, instead of hook and/or loop material, the attachment mechanism can include hook compatible material, hook compatible web or combinations thereof. Such materials can provide improved adjustability, in some applications, it is understood that other apparatus for connecting each end of the anterior strap 203 are possible and can include, but is not limited to, no apparatus on the strap and double loops attached to the cervical traction device, strap adjusters (FIG. 4), adjustable strap latches, slip rings or combinations thereof.

In certain examples, the optional cushion 208 can include a cushion insert and an external material to cover the cushion insert. In certain examples, the cushion 208 can be attached to the web 213 of the anterior strap 203 such as by sewing the cushion 208 to the web. In some examples, the web 213 can include an area of hook and loop material to attach a cushion 208 and the cushion 208 can include an corresponding area of hook and loop material. In certain examples, a corresponding area of hook and loop material can be attached to the external material of the cushion 208 to allow the cushion to be single-use and to easily be replaced. In some examples, external neoprene material can be hook and loop compatible for easy replacement of the cushion 208.

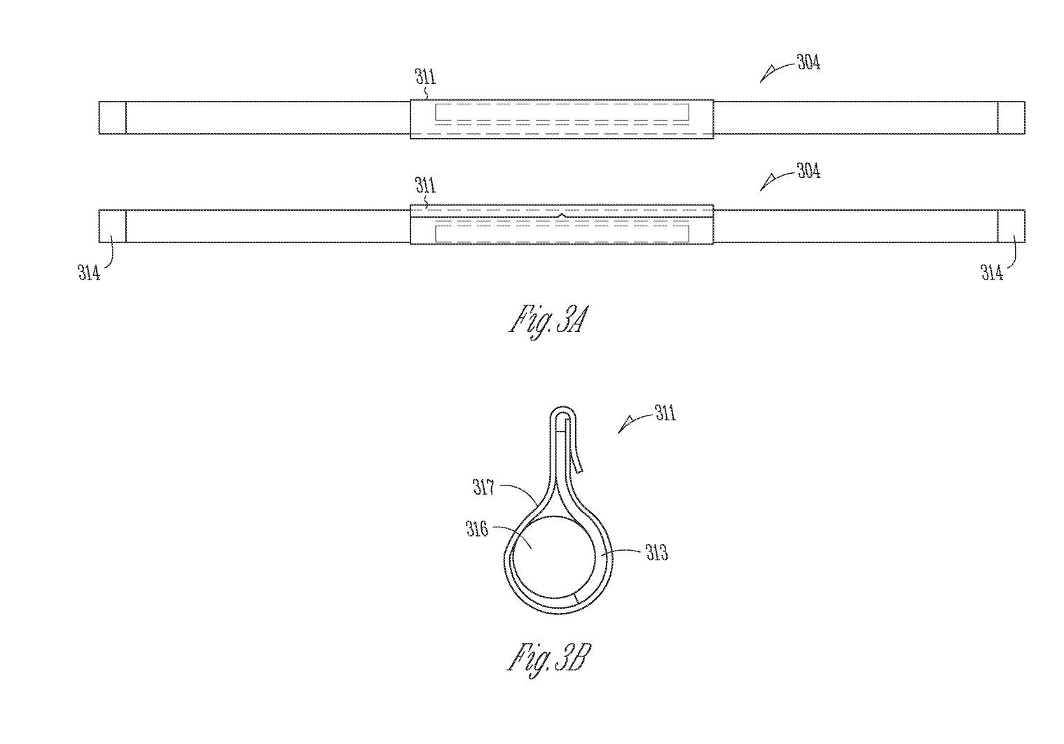

FIGS. 3A and 3B illustrate generally a posterior strap 304 for a cervical traction device according to an example of the present subject matter. FIG. 3A illustrate generally top and bottom views of the posterior strap 304. In certain examples, the posterior strap 304 can include a web 313 and an occipital bone interface 311. In certain examples, the web can define the length of the posterior strap 304. In certain examples, the occipital bone interface 311 is centrally located along the length of the posterior strap 304 and has a length to fit a range of subjects. In certain examples, the web 313 can pass continuously along the entire length of the posterior strap 304, In some examples, a first length of web 313 can couple to a first end of the occipital bone interface 311and a second length of web 313 can be coupled to a second end of the occipital bone interface 311. In certain examples, each end of the posterior strap 304 includes an attachment apparatus for adjustably securing the strap 304 to a cervical traction device. In certain examples, the posterior strap 304 can include two areas of corresponding book and loop material at each end of the posterior strap 304. Such an arrangement of hook and loop material can allow an end of the strap 304 with the first area of book and loop material to pass through a loop anchor and then fold back on the corresponding second area of hook and loop material. The lengths of the areas of hook and loop material can define the limits of adjustment of the posterior strap. In some examples, each end of the posterior strap 304 can include only one area of hook and loop material 314 and the posterior strap 304 can be secured using a second corresponding area of hook and loop material attached to the cervical traction device. In some examples, instead of hook and/or loop material, the attachment mechanism can include hook compatible material, hook compatible web or combinations thereof. Such materials can provide improved adjustability, in some applications. It is understood that other apparatus for connecting each end of the posterior strap are possible and can include, but is not limited to no apparatus on the strap and double loops attached to the cervical traction device, strap adjusters (FIG. 4). adjustable strap latches or combinations thereof.

FIG. 3B illustrates a cross section view of an occipital bone interface 311 of a posterior strap 304. In certain examples, the occipital bone interface 311 can include a web 313, a tubular insert 316 and an exterior material 317. In certain examples the web is a length of polypropylene web that can define the overall length of the posterior strap 304. It is understood that other strap materials can be used in place of the web 304 without departing from the scope of the present subject matter. In certain examples, the tubular insert 316 can include a foam tube such as an extruded foam tube. The size of the tubular insert 316 can vary and can depend on the specific structure of the subject near the base of the occipital bone, In some examples, the diameter of the tubular insert 316 can be about ½ to about 2″. In some examples, a user can select a tubular insert 316 from a set of tubular inserts that vary in ¼ increments from ½ to about 2″. Materials other than foam can be used for the tubular insert 316 without departing from the scope of the present subject matter and can include, but are not limited to, rubber, plastic, gel packs, microbeads or combinations thereof. In general, the tubular insert 316 should provide some give or softness for comfort yet be firm enough to interface with the occipital bone and remain in position without slipping during application of traction force.

In certain examples, the exterior material 317 can be a fabric that is comfortable for a subject to apply to the skin and can be sewn or otherwise asserribled with the web 313 and tubular insert 316. In certain examples, the external material 317 can include a rubberized external surface to provide grip or stick-type friction when positioned to interface with a subject's occipital bone under traction force,

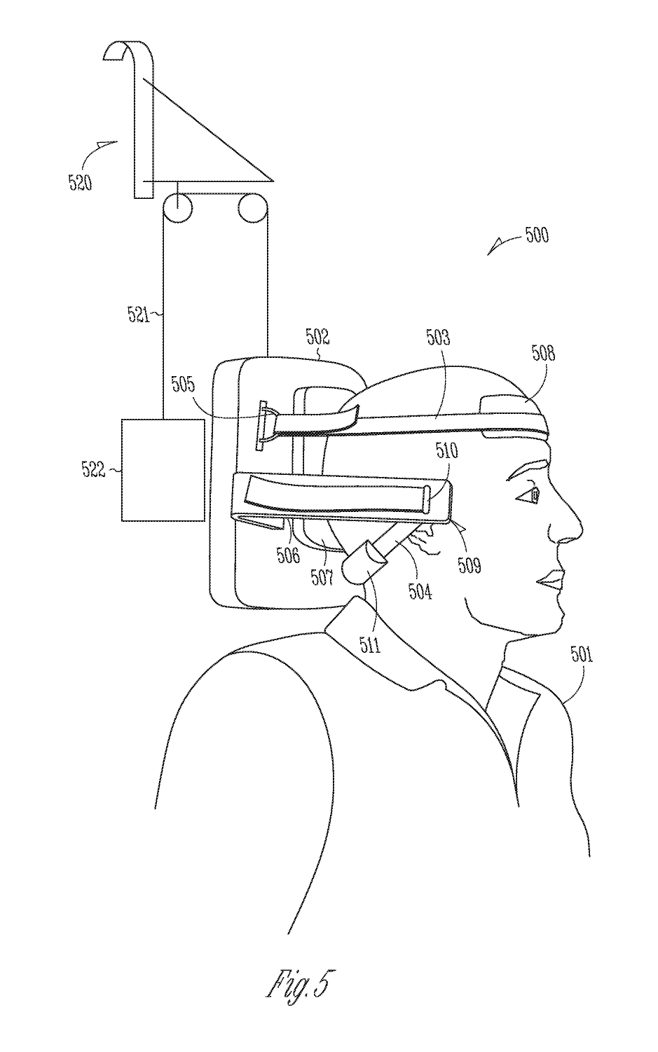

FIG. 5 illustrates generally use of a cervical traction device according to an example of the present subject matter on a subject in an upright position. In certain examples, the cervical traction device 500 can include a hanger assembly 520 including traction weight 522, a traction base 502, an anterior strap 503 and a posterior strap 504. The traction base 502 can provide a rigid structure to anchor forces applied to the subject 501 via the straps 503, 504. In certain examples, the traction base 502 can include left and right 505 anterior strap anchors, left and right 506 posterior anchor structures, and one or more optional cushions 507 to support the back of the head of a subject 501.

The left anterior strap anchor (not shown) and the right anterior strap anchor 505 can allow an anterior strap 503 to be laid across the frontal bone of the subject 501 and secured to the traction base 502. In certain examples, the anterior strap 503 can include hook and loop material (not shown) on a major surface of the strap 503 near each end of the anterior strap 503. Corresponding hook and loop material can be located on the same major surface closer to the center of the anterior strap 503. Each end of the anterior strap 503 can be secured to the corresponding left or right anterior strap anchor 505 by passing the end of the anterior strap 503 through the loop of the anchor 505 and folding the strap 503 back on itself and coupling together the hook and loop material at the end of the strap 503 to the corresponding hook and loop material located closer to the center of the strap 503. In certain examples, the anterior strap 503 can include a comfort pad 508 that, in certain examples, can be positioned between the strap 503 and the subject's skin. In certain examples, the traction base 502 can include multiple pairs of anterior strap anchors 505 to accommodate subjects with different sized heads. In some examples, a single pair of anterior strap anchors 505 can provided and can be adjustable to accommodate the size and shape of the head of each individual subject.

The left posterior anchor structure (not shown) and the right posterior anchor structure 506 can allow the posterior strap 504 to be positioned and secured such that the posterior strap 504 provides an appropriate cervical traction force. The posterior anchor structures 506 can he mechanically coupled to the traction base 502 and can provide an anchor position 509 near the subject's temples such that the posterior strap 504 can exert a traction force that can stretch the neck muscles, provide a separation force to the vertebra at the top of the spine, open the nerve channels of the spine near the neck or combinations thereof. In some examples, the posterior anchor structures 506 can be adjustable. In some examples, the posterior anchor structures 506 can be adjustable along an axis parallel to the subject's spine. In some examples, the posterior anchor structures 506 can be adjustable along an axis perpendicular to the subject's spine. In some examples, the posterior anchor structures 506 can be adjustable along an axis perpendicular to the subject's spine and along an axis perpendicular to the subject's spine to accommodate subjects of different size and shape.

In certain examples, each posterior anchor structure 506 can include an anchor apparatus for interfacing the posterior strap 504 with the posterior anchor structure 506. In some examples, the anchor apparatus can he a loop. In the illustrated example of FIG. 5, the anchor apparatus includes a slot 510 through which a corresponding end of the posterior strap 504 can be routed, Thus, the center portion 511 of the posterior strap 504 can be placed at the base of the occipital bone of the subject and the ends of the posterior strap 504 can be routed through the slots 510 of each posterior anchor structure 506. In certain examples, each end of the posterior strap 504 can include a length of hook and loop material. In some examples, a corresponding length of hook and loop material can be attached to an outer surface of each posterior anchor structure 506 and the posterior strap can be secured in place by mating the hook and loop material at the each end of the strap 504 with the hook and loop material attached to the corresponding posterior anchor structure 506. It is understood that strap hardware other than hook and loop material, loop anchors, and slots can be used to provide adjustable anchoring for the anterior strap 503 and the posterior strap 504 without departing from the subject matter of the present subject matter. It is also understood that the shape and size of the traction base 502 and the right and left posterior anchor structures 506 can differ from that shown in FIG. 5 without departing from the scope of the present subject matter.

In certain examples, the subject can stand or sit near a door and the hanger assembly can be used to position the cervical traction device in place to position and secure the anterior strap 503 and the posterior strap 504 about the subject's frontal bone/forehead and occipital bone respectively. A traction weight 522 can then be secured to a cable 521 passing through the hanger assembly 520 and connected to the traction base 502. As the traction weight 522 is allowed to be suspended by the cable, traction force can be applied to the subject via the traction base 502, anterior strap 503, and posterior strap 504.

In certain examples, the traction base 502 can be mounted to a slide mechanism (not shown) to allow the traction base 502 to move in a vertical direction, yet reduces the possibility of the traction base 502 rotating in the vertical plane. In some examples, the slide mechanism can include linear guides and the traction base 502 can include linear bearings that slide along and are mechanically captured by the linear guides. In some examples, the traction base 502 can include the linear guides and the slider mechanism can include the linear bearings. In certain examples, the slider mechanism can be fastened to the door, the chair the user is seated in, or other object that user is using or that the hanger assembly is hung from.

### ADDITIONAL EXAMPLES AND NOTES

In Example 1, a cervical traction device can a traction base, an anterior strap configured to apply pressure to a frontal bone of the head and to couple to the traction base, and a posterior strap configured to engage an occipital bone of the subject and to couple to the traction base, wherein the posterior strap includes a tubular portion configured to engage the occipital bone.

In Example 2, the device of Example 1 optionally includes a pair of posterior anchor structures coupled directly to the base and configured to provide an anchor position for each end of the posterior strap.

In Example 3, the pair of posterior anchor structures of any one or more of Examples 1-2 optionally is adjustable along an axis parallel to the subject's spine when the subject is oriented for engagement of the anterior strap and the posterior strap.

In Example 4, the pair of posterior anchor structures of any one or more of Examples 1-3 optionally is adjustable along an axis perpendicular to the subject's spine when the subject is oriented for engagement of the anterior strap and the posterior strap.

In Example 5, each posterior anchor structure of the pair of posterior anchor structures of any one or more of Examples 1-4 optionally is individually adjustable along an axis parallel to the subject's spine when the subject is oriented for engagement of the anterior strap and the posterior strap.

In Example 6, each posterior anchor structure of the pair of posterior anchor structures of any one or more of Examples 1-5 optionally is individually adjustable along an axis perpendicular to the subject's spine when the subject is oriented for engagement of the anterior strap and the posterior strap.

In Example 7, the pair of posterior anchor structures of any one or more of Examples 1-6 optionally is adjustable along an axis parallel to the subject's spine and along an axis perpendicular to the subject's spine when the subject is oriented for engagement of the anterior strap and the posterior strap.

In Example 8, each posterior anchor structure of any one or more of Examples 1-7 optionally includes a slot, distal from the traction base, to define the anchor position for each end of the posterior strap.

In Example 9, the posterior strap of any one or more of Examples 1-8 optionally includes a first length of hook and loop material near each end, and each posterior anchor structure of any one or more of Examples 1-8 optionally includes a corresponding second length of hook and loop material to which the first length is configured to interact and secure the posterior strap.

In Example 10, the posterior strap of any one or more of Examples 1-9 optionally includes a tubular insert and an outer layer of fabric enclosing the tubular insert.

In Example 11, the tubular portion of the posterior strap of any one or more of Examples 1-10 optionally includes a raised circular cross section configured to engage a base of the occipital bone of the subject and to allow traction force to be comfortably and effectively applied to the subject.

In Example 12, the anterior strap of any one or more of Examples 1-1 optionally includes a first major surface, a second major surface opposite the first major surface, and a forehead pad attached to the first major surface and centrally located along the length of the anterior strap.

In Example 13, the traction base of any one or more of Examples 1-12 optionally includes a first pair of anchors configured to secure the anterior strap to the traction base.

In Example 14, the anterior strap of any one or more of Examples 1-13 optionally include a first length of hook and loop material near each end, and a corresponding second length of hook and loop material adjacent each first length, each corresponding second length configured to interact with one of the first lengths and secure an end of the anterior strap to one of the anchors of the pair of anchors.

In Example 15, the device of any one or more of Examples 1-14 optionally includes a second pair of anchors configured to secure the posterior strap to the traction base.

In Example 16, the device of any one or more of Examples 1-15 optionally includes a hanger coupled to the traction base, wherein the hanger and traction base are configured to provide cervical traction when the spine of the subject is vertical.

In Example 17, the posterior strap of any one or more of Examples 1-16 optionally is configured to not apply a traction force to a temporomandibular joint (TMJ) of the subject.

Each of these non-limiting examples can stand on its own, or can be combined with one or more of the other examples in any permutation or combination.

The above detailed description includes references to the accompanying drawings, which form a part of the detailed description, The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure, It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment, Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are legally entitled.

What is claimed is:

1. A cervical traction device configured to provide cervical traction to a subject using the head of the subject, the cervical traction device comprising:
a traction base;
an anterior strap configured to apply pressure to a frontal bone of the head and to couple to the traction base; and
a posterior strap configured to engage an occipital bone of the subject and to couple to the traction base, wherein the posterior strap includes a tubular portion configured to engage the occipital bone.

2. The device of claim 1, including a pair of posterior anchor structures coupled directly to the base and configured to provide an anchor position for each end of the posterior strap.

3. The device of claim 2, wherein the pair of posterior anchor structures is adjustable along an axis parallel to the subject's spine when the subject is oriented for engagement of the anterior strap and the posterior strap.

4. The device of claim 2, wherein the pair of posterior anchor structures is adjustable along an axis perpendicular to the subject's spine when the subject is oriented for engagement of the anterior strap and the posterior strap.

5. The device of claim 2, wherein each posterior anchor structure of the pair of posterior anchor structures is individually adjustable along an axis parallel to the subject's spine when the subject is oriented for engagement of the anterior strap and the posterior strap.

6. The device of claim 2, wherein each posterior anchor structure of the pair of posterior anchor structures is individually adjustable along an axis perpendicular to the subject's spine when the subject is oriented for engagement of the anterior strap and the posterior strap.

7. The device of claim 2, wherein the pair of posterior anchor structures is adjustable along an axis parallel to the subject's spine and along an axis perpendicular to the subject's spine when the subject is oriented for engagement of the anterior strap and the posterior strap.

8. The device of claim 2, wherein each posterior anchor structures includes a slot, distal from the traction base, to define the anchor position for each end of the posterior strap.

9. The device of claim 8, wherein the posterior strap include a first length of hoof and loop material near each end; and
wherein each posterior anchor structure includes a corresponding second length of hook and loop material to which the first length is configured to interact and secure the posterior strap.

10. The device of claim 1, wherein the posterior strap includes:
a tubular insert; and
an outer layer of fabric enclosing the tubular insert.

11. The device of claim 10, wherein the tubular portion of the posterior strap includes a raised circular cross section configured to engage a base of the occipital bone of the subject and to allow traction force to be comfortably and effectively applied to the subject.

12. The device of claim 1, wherein the a strap includes:
a first major surface;
a second major surface opposite the first major surface; and
a forehead pad attached to the first major surface and centrally located along the length of the anterior strap.

13. The device of claim 1, wherein the traction base includes:
a first pair of anchors configured to secure the anterior strap to the traction base.

14. The device of claim 13, wherein the anterior strap include a first length of hook and loop material near each end; and
corresponding second lengths of hook and loop material adjacent each first length, each corresponding second length configured to interact with one of the first lengths and secure an end of the anterior strap to one of the anchors of the pair of anchors.

15. The device of claim 14, including a second pair of anchors configured to secure the posterior strap to the traction base.

16. The device of claim 1, including a hanger coupled to the traction base, wherein the hanger and traction base are configured to provide cervical traction when the spine of the subject is vertical.

17. The device of claim 1, wherein the posterior strap is configured to not apply a traction force to a temporomandibular joint (TMJ) of the subject.
